(19) United States
(12) Patent Application Publication
(10) Pub. No.: US 2021/0346378 A1
(43) Pub. Date: Nov. 11, 2021

(54) METHOD TO PREVENT AND TREAT ALOPECIA BY CALCIUM CHANNEL BLOCKERS, ANGIOTENSIN CONVERTING ENZYME INHIBITORS, AND ANGIOTENSIN RECEPTOR BLOCKERS

(71) Applicant: Assa Weinberg, Los Angeles, CA (US)

(72) Inventor: Assa Weinberg, Los Angeles, CA (US)

(21) Appl. No.: 17/382,624

(22) Filed: Jul. 22, 2021

Related U.S. Application Data
(63) Continuation of application No. PCT/US2020/014445, filed on Jan. 21, 2020.
(60) Provisional application No. 62/796,335, filed on Jan. 24, 2019.
(60) Provisional application No. 62/796,341, filed on Jan. 24, 2019.
(60) Provisional application No. 62/796,352, filed on Jan. 24, 2019.

(51) Int. Cl. A61K 31/502; A61K 31/41; A61K 31/216; A61P 17/14; A61K 9/00

(52) U.S. Cl. CPC A61K 31/502; A61K 31/41; A61K 9/0014; A61P 17/14; A61K 31/216

(57) ABSTRACT

A method is provided to prevent and to treat alopecia by using Calcium Channel Blockers (CCB), Angiotensin-Converting Enzyme (ACE) Inhibitors, or Angiotensin Receptor Blockers (ARB), and more particularly, to a method to prevent and treat alopecia by using Calcium Channel Blockers, Angiotensin-Converting Enzyme Inhibitors, or Angiotensin Receptor Blockers that are not taken orally, but administered through the skin into the scalp space to form contact with the scalp skin where hair loss is present to treat and to heal hair loss.

### INCORPORATION BY REFERENCE TO RELATED APPLICATION

This application is a continuation of PCT Application No. PCT/US2020/014445, filed Jan. 21, 2020, which claims the benefit of U.S. Provisional Application No. 62/796335, filed Jan. 24, 2019, U.S. Provisional Application No. 62/796341, filed Jan. 24, 2019, and U.S. Provisional Application No. 62/796352, filed Jan. 24, 2019. Each of the aforementioned applications is incorporated by reference herein in its entirety, and each is hereby expressly made a part of this specification.

### FIELD OF THE INVENTION

A method is provided to prevent and to treat alopecia by using Calcium Channel Blockers (CCB), Angiotensin-Converting Enzyme (ACE) Inhibitors, or Angiotensin Receptor Blockers (ARB), and more particularly, to a method to prevent and treat alopecia by using Calcium Channel Blockers, Angiotensin-Converting Enzyme Inhibitors, or Angiotensin Receptor Blockers that are not taken orally, but administered through the skin into the scalp space to form contact with the scalp skin where hair loss is present to treat and to heal hair loss.

### BACKGROUND OF THE INVENTION

Hair loss is among the most common human complaints worldwide affecting both men and women alike. While diagnosis can be easily done by nonprofessionals, the cause of hair loss, and in particular, the treatment, remains a professional challenge.

A hair follicle has two regions: the extra dermal and intra dermal regions. The visible part of the hair follicle is made of keratin, a hard pigmented protein. The intra dermal part of the hair follicle called is the dermal papilla region. There are 100000 to 150000 hair follicles in human scalp.

Anatomically the dermal papilla itself is comprised of four segments. The bulb, the supra bulb, the isthmus, and the infundibulum. The bulb, the base of the papilla, is composed of a layer of keratinocytes. Keratinocytes are specialized cells that produce the hair core protein, keratin. The supra bulbar part is the segment that separates the bulb from the isthmus. The isthmus is the area that comprises the extra hair apparatus, the sebaceous gland and the hair erector muscle. The infundibulum is the area that extends from the sebaceous gland to the skin surface.

The hair shaft structure was the subject of extensive studies. It is made of three different layers of keratins. In the innermost layer, the medulla is surrounded by a middle layer the-cortex. The cortex extends from the bulb to the isthmus only, the outer layer, the cuticle, hugs the hair shaft from the bulbs to the skin surface.

More than 57 genes determine the keratin structure and pigmentation and are responsible for the large spectrum of human hair shades and colors. Recently, a new region was discovered and added to the three traditional segments of hair follicle. It is localized in the vicinity of the erector pili attachment to the supra bulbar shaft. Because of its shape it is referred to as the bulge region. Its importance is attributed to the fact that it contains the hair follicles stem cells and is responsible for hair growth cycle and replacement. Loss of the bulge region is now considered the main cause of irreversible hair loss or cicatricial alopecia.

The human body possesses two types of hair follicles: vellus hair and terminal hair. Vellus hair are short, imbedded in the dermal reticular layer of the skin, with a diameter of less than 0.003 mm. Terminal hair are imbedded in the subcutaneous fat layer and are long, thicker, with a diameter of about 0.006 mm. Other hair with intermediate diameter between 0.003 and 0.006 were identified as well. At birth, vellus hair occupy the majority of skin bearing area. Terminal hair is exclusively found in the scalp, eyebrows, and eyelashes. In puberty the vellus hairs in the genital area and the axillary territories undergo conversion to terminal hair. Conversion of vellus hair in other territories to terminal hair is the cause of female hirsutism. Terminal hair conversion to vellus hair underscore female androgenic alopecia.

Once the final genetic structures are achieved, terminal hair and vellus hair undergo lifelong cycles of growth (anagen), and rest, (telogen) separated by a short transitional follicular shrinking, (catagen) this cycle affects only the papilla, and are not synchronized. Only 50-150 telogen hairs are shed per day, to prevent massive hair loss and a complete exposure of skin and scalp territory. At any time 90% of the hair is at the anagen phase, 10% are at the telogen phase, and less than 1% is at the catagen phase. The anagen period determines hair length. Eyebrow and eyelashes are terminal hair with 0.1 diameter hair with an anagen phase that is restricted two to three months. That limits their length to 6 to 9 mm. Vellus hair, with similar diameter of 0.1 mm, can reach a length of 20 mm because of a longer anagen phase of 3-6 months. Scalp terminal hair with diameter of 0.3 mm with an anagen phase of two to six years can reach 7-20 cm in length. The telogen phase is a resting period. At the end of the rest period the hair above the isthmus sheds. The shed hair is distinguished by its fully keratinized golf club shape.

The reversibility of hair loss is the basis of the medical classification of all alopecia. Irreversible hair loss is called cicatricial hair loss. The reversible ones are called non cicatricial alopecia. Non cicatricial alopecia are diseases where spontaneous recovery is observed, or currently available treatment can resume cellular follicular hair keratin synthesis production and hair growth. Recent studies conferred this revival ability to the transformation of hair stem cells in the follicle bulge region to mature pulp keratinocytes. The cicatricial alopecia are diseases where spontaneous recovery is never observed. In addition, currently available treatment can not resuscitate the follicular hair cells to renew keratin synthesis and resume hair growth. Such disease process puts a permanent stop to the anagen and telogen cycles, and cause loss of bulge region stem cells which ends in follicular hair death.

Cicatricial alopecia are a group of chronic inflammatory diseases caused by scalp invasion by inflammatory hair killer cells. The cellular type of the invasive cells is the current basis of hair loss disease classifications. They are: diseases caused by neutrophilic cells invasion; diseases caused by lymphocytic cells invasion; and and the mixed cell types alopecia diseases.

Two disease entities are included in the neutrophilic group. These include the dissecting cellulitis of the scalp, a form of painful hair loss produced by very inflamed hair follicles. The disease usually affects young Africans American men. Aggressive therapy that includes surgical resection of the inflamed follicles, x ray radiation, and skin grafting produced results only in the minority of patients. Folliculitis decalvans is distinguished by the appearance of multiple hairs emerging from a single hair follicle.

Primary lymphocytic cicatricial alopecia contains a long list of diseases that include, among others, such syndromes as: discoid lupus erythematosis, a hair loss that leads to atrophic scars and visible dilated capillaries; lichen plan, is painful, symptomatic patchy hair loss caused by sensitive scalp skin; frontal fibrosing alopecia, consisting of frontal scalp band of hair loss associated with eyebrow hair as well; and pseudopelads of Brock, a hair loss that resembles a footprint in the snow that affects the occipital hair.

Mixed cellular alopecia involves hair loss associated with two distinct forms of acne: acne necrotica, that leaves pox like scars on the scalp; and occipital hair loss affecting young African Americans that lead to the formation of keloids on the occipital scalp. This kind of hair loss is called Acne Koloidalis Nucha.

Diseases that cause reversible alopecia are clinically characterized by the absence of the symptoms which are common in irreversible hair loss diseases, such as pain, pruritus, and erythema. Microscopically, the hair follicle structure in reversible alopecia is intact.

The diagnostic clue of reversible hair loss diseases is frequently delivered by the pattern and the topographic locations of the alopecia. The current reversible hair loss disease classification includes three such categories: Focal, diffuse and patterned hair loss.

Five focal alopecia are described. Alopecia aerata, a common scalp or occipital shiny hair loss that appears over few weeks. It is caused by an auto immune process and genetic. Low 25 hydroxy vitamin D serum level was recently found to correspond to the disease severity. Occasionally it can appear in a diffuse pattern. Both forms respond to intra lesions glucocorticoid injection treatment. Alopecia syphillitica, secondary syphilis, can produce the so called moth eaten alopecia. Hair loss that involve scalp eyebrow and beard, and is reversible with treatment. Post operative induced alopecia, temporal triangular alopecia, and hair dressing produced alopecia, known as traction alopecia, are other forms of focal and reversible hair loss.

Pattern alopecia includes three diseases that affects millions of people worldwide. Male androgenic alopecia is age related. Among white men, it affects 16% by the age of 15, 30% by the age of 30, and 80% of age above 80. African Americans and Asians are substantially less affected. Dehydrotestosterone (DHT) is the key hormone responsible for hair loss in men, and is multiple times more balding than testosterone. DHT is converted from testosterone by a 5 alpha reductase enzymatic activity. The enzyme is abundantly located in the hair follicles of men's temporal scalp areas, where alopecia takes place. The enzyme has two forms. Type one is Intradermal, attached to hair structure from the papilla to the sebaceous gland. Type two is attached to the outer sheet of the hair in the epidermis. DHT binds to the hair cell nuclear androgen receptor complex. There it activates the genes that transform the large hair follicles to miniaturized vellus hair follicles. In the smaller size follicles, the duration of the anagen phase is shortened from six years to three months.

The visual progression of alopecia in men can be assigned to 7 successive photographic stages that are the basis for the Hamilton Norwood alopecia scale, which outlines a distinct progression of balding. Hamilton Norwood scale describes 90% of male hair loss pattern. The remaining 10% of male alopecia involves the Christmas tree pattern, typical of female hair

Loss. Medical treatment of men androgenic alopecia is comprised of two medications only. Finasteride and Minoxidil. Finasteride is the 5 alpha reductase enzyme blocker, the hair shaft enzyme that converts testosterone to DHT. Hair follicle miniaturization agent. Its use is based on multiple clinical studies.

Minoxidil is the most commonly used drugs for men androgenic baldness in the US. It is available in three pharmaceutical forms and does not require prescription in the U.S.: 2% and 5% solutions, and the 5% foam. Minoxidil activity in hair cells was extensively studied. It reverses the DHT shortening of the anagen growth phase from 3-6 months back to 3-6 years, and propels the miniaturized vellus hair papilla to form the large terminal hair-follicles size. Multiple clinical trials justify the use of Minoxidil. The largest trial, a superiority trial type, between the 5% to the 2% showed that after 48 weeks the 5% solution generated 18.6 terminal hair compared to 12.7 terminal hair by the 2% solution per square centimeter.

The human female gender is also afflicted by age related hair loss. In the past this phenomenon appeared under different names but now is known as female pattern hair loss. Like in men, age is the major determinant factor for female hair loss. 3% at age 20 and 39% by age 70. Female hair loss was found to reach 39% of women above age 70 in a British study. The pattern of age related women hair loss involves thinning of hair in different scalp territories than age related men hair loss. It spares the temporal areas and affects the frontal and vertex areas to give the appearance of a Christmas tree. It also spares the occipital area. The mechanisms for the Christmas tree pattern hair thinning of female alopecia involve a shortening of anagen growth phase from six years to three months, and transformation of the large terminal scalp hair follicles to a miniaturized vellus hair follicles.

Like the age related male pattern alopecia, the age related female pattern hair loss is linked to an increase in diabetes, hypertension, and an increase in cardiovascular mortality risks.

The treatment of age related female pattern hair loss is similar to the age related hair loss pattern in men. Finasteride, the five alpha reductase blocker that prevents the conversion of testosterone to DHT is commonly used. The three non prescription forms of Minoxidil: 2% and 5% solutions, and the 5% foam are the first line of treatment. Their use is based on multiple clinical studies. It takes 4 months before a visible effect can be observed, and 12 months is the usual recommended treatment period.

Minoxidil is a potent vasodilator. This activity is achieved by a dynamic, successive cascade of changes in intracellular ions. Current knowledge of Minoxidil molecular hair growing activity are based on cell culture experiments. Minoxidil is a potassium channel opener. The opening of these channels causes potassium out flux. Such flow generates increase in the concentration of the intra cellular cytoplasmic calcium. The increase in calcium concentration leads to increase in cellular adenosine. The higher level of adenosine triggers the production of vascular endothelial growth factor, a promoter of hair growth. Other cell culture experiments strengthen the Minoxidil adenosine VEGF concept. The addition of increasing concentration of Minoxidil to papilla dermal cell culture produced similarly increased concentration of VEGF mRNA. VEGF concentrations are high during the anagen phase and low during the telogen phase.

In spite of the clarity of the adenosine dependent Minoxidil hair growing activity, it has only been confirmed in follicular papilla cell culture. It is still unclear whether potassium channels are expressed in hair follicles.

Dermal papilla cells have highly developed capillary network. Surprisingly the activity of other and safer vasodilators was never studied in hair loss. Side effects of current treatment of androgenic alopecia include the following. Finasteride: affects sexual function. The side effects include: decreased libido, erectile dysfunction and ejaculatory dysfunction. Minoxidil: contact dermatitis, irritant dermatitis. Minoxidil side effects of treatment of female pattern hair loss include contact dermatitis, irritable dermatitis, and facial hypertrichosis. While hirsutism is not a problem in men, it is a problem in women. Facial hair growth during Minoxidil treatment is a major side effect, which takes at least 4 months to resolve.

For efficacy of Minoxidil treatment, pool data of 6 minoxidil trials in women showed that only 25.8% (157/593) of participants reported a moderate to marked increase in hair growth. Trial results of Minoxidil against placebo are different in men than in women. Results are four times better than placebo. These figures are misleading. Acceptable cosmetic results are achieved only in 30% of chronic Minoxidil users.

The efficacy of Finasteride was assessed by meta analysis of 12 studies. After one year of treatment, hair count was 9.42% higher than baseline in Finasteride as compared to placebo. And 24.3% after 4 years. Results were considered moderate. Even this moderate results are better than what can be achieved by Minoxidil. As was demonstrated by the only 5mg finasteride versus Minoxidil comparative study. Even against a lower dose Finasteride, results of Minoxidil 2% one-year treatment were significantly inferior in hair count. The cosmetic results of both were considered non-statistically different. Finasteride's higher potency in hair growth is associated with more serious side effects than Minoxidil. It can cause reduction in sperm count, gynecomastia, testicular pain and depression, and adulterous test results of the prostatic cancer detection test PSA. Prolonged use of Finasteride is associated with reduction in prostatic cancer incidence but a significant increase in a more malignant form of this disease.

### SUMMARY OF THE INVENTION

As is clear from the above discussion, the data highlights the limitation of the current mode of treatment and the lack of preventive treatment of male hair loss, and in particular, the limited options of prevention and treatment for age related male hair loss.

This data highlights the limitation of the current mode of treatment and the current lack of preventive treatment for female hair loss, and in particular, the limited options of prevention and treatment of age related female hair loss.

There is a need for a new mode of prevention and a new mode of treatment for hair loss, and in particular, a new mode of prevention and treatment for the age related men and women hair loss.

Additionally, the embodiments use a method of administering by application, of a pharmacological composition according to the invention in an effective amount, of a calcium channel blocker, an angiotensin-converting enzyme inhibitor, or an angiotensin receptor blocker, directly to scalp, where alopecia and alopecia symptoms are located.

Pharmacological preparation as used herein is a pharmacological preparation according to the invention, composed but not limited to, a calcium channel blocker, an angiotensin-converting enzyme inhibitor, or an angiotensin receptor blocker, and a suitable nontoxic pharmacological carrier.

Effective amount as used herein is an amount of the pharmaceutical composition which is effective for treating hair loss. An amount of a calcium channel blocker, an angiotensin-converting enzyme inhibitor, or an angiotensin receptor blocker that is suitable for treatment of alopecia.

Contact calcium channel blockers, angiotensin-converting enzyme inhibitors, and angiotensin receptor blockers are a new pharmaceutical class of medications that increase the capillary network and augment the blood supply to the scalp epidermis to prevent the process and symptoms of hair loss.

For the foregoing reasons, there is a need for a method to treat hair loss by increasing the capillary network and augment the blood supply to, but not restricted to: the scalp, the scalp epidermis and other soft tissues associated with alopecia and alopecia symptoms.

For the forgoing reason there is a need for a method to prevent alopecia and the symptoms associated with alopecia by increasing the capillary network and augment the capillary blood supply to scalp territories where alopecia is known to be formed, and in particular to the scalp territories of age related male pattern alopecia, and of age related female pattern alopecia.

The present embodiments are directed to a method for preventing alopecia of the scalp epidermis by direct application to the scalp epidermis of a calcium channel blocker, an angiotensin-converting enzyme inhibitor, or an angiotensin receptor blocker to prevent the formation and symptomatology of alopecia.

By “scalp alopecia” as used herein, it is meant any alopecia of the of the scalp epidermis that causes the symptoms of alopecia.

In addition the embodiments are directed to the treatment of alopecia caused by, but not limited to, premature hair loss, hair loss by medications that block the hair growth activity, hair growth failure, radiation or other causes that cause alopecia and alopecia symptoms.

In one method of the invention a calcium channel blocker, an angiotensin-converting enzyme inhibitor, or an angiotensin receptor blocker may be applied directly to the scalp epidermis before symptoms form to prevent alopecia, for example, in women or men who have normal hair count before the onset of alopecia symptoms.

In another embodiment of the invention, a calcium channel blocker, an angiotensin-converting enzyme inhibitor, or an angiotensin receptor blocker may be applied directly to the scalp epidermis to treat and heal the symptoms of alopecia. Calcium channel blockers, angiotensin-converting enzyme inhibitors, and angiotensin receptor blockers may be applied even after the alopecia syndrome has dissipated and the scalp epidermis has regrown hair, to prevent the recurrence of alopecia and alopecia related syndromes.

Accordingly, in a generally applicable first aspect (i.e., independently combinable with any of the aspects or embodiments identified herein), the Calcium Channel Blocker, ACE Inhibitor, or Angiotensin Receptor Blocker may be administered directly to the scalp before symptoms form to prevent alopecia. For example, in men or women with normal hair growth before symptoms of alopecia appear.

In a generally applicable embodiment (i.e., independently combinable with any of the aspects or embodiments identified herein) of the first aspect, the Calcium Channel Blocker, ACE Inhibitor, or Angiotensin Receptor Blocker may be administered directly to the scalp to treat and heal the symptoms of alopecia.

In a generally applicable embodiment (i.e., independently combinable with any of the aspects or embodiments identified herein) of the first aspect, the Calcium Channel Blocker, ACE Inhibitor, or Angiotensin Receptor Blocker may be administered even after the alopecia has been treated to prevent recurrence.

Calcium Channel Blockers, ACE Inhibitors, and Angiotensin Receptor Blockers are classes of pharmaceutical drugs when taken orally they dilate the arteriolar system by blocking the activity of the calcium channel receptors, ACE receptors or angiotensin receptors.

Contact Calcium Channel Blockers, ACE Inhibitors, and Angiotensin Receptor Blockers are a new class of pharmaceutical products. When applied directly to target tissue they increase the capillary network, augment the capillary blood supply, and enhance tissue repair in diverse body membrane tissues.

Currently, there are over 20 pharmaceutical patented ACE Inhibitor or Angiotensin Receptor Blocker drugs that use this property to treat hypertension, and congestive heart failure. The clinical indication of ACE Inhibitors or Angiotensin Receptor Blockers is therefore currently limited to the field of cardiovascular diseases. Calcium channel blockers are a class of pharmaceutical drugs that disrupt the entry of calcium molecules through the L type voltage operated channels to cardiac muscle and blood vessels cells. The blockage of calcium entry causes relief of arterial spasm. Currently there are 70 pharmaceutical patented calcium channel blocker drugs that use this property to treat hypertension, angina pectoris and cardiac arrhythmia. The clinical indication of calcium channel blockers is therefore also limited to the field of cardiovascular diseases.

Calcium channel blockers, ACE inhibitors, and angiotensin receptor blockers were extensively studied but their ability to prevent and treat alopecia remained unknown. No trial of topical calcium channel blockers, ACE inhibitors, or angiotensin receptor blockers for the prevention and treatment of alopecia was ever published.

The use of application of Calcium Channel Blockers, ACE Inhibitors or Angiotensin Receptor Blockers to the scalp is provided. The direct contact with the scalp tissue increases the capillary network and augments the blood supply to the area to facilitate hair growth or regrowth.

The new class may be used for the prevention and treatment of alopecia.

### DETAILED DESCRIPTION OF THE INVENTION

The following discussion addresses a number of embodiments and applications of the present disclosure. The beneficial features of the present disclosure will be evident from the described embodiments. It is to be understood that the present disclosure is not limited to such specific applications and that numerous implementations of the present disclosure may be realized. All references to patents, patent applications, and non-patent publications mentioned in the specification are hereby incorporated by reference, in their entireties.

Contact neo-vasodilators are a new class of medication. This invention describes the use of contact neo-vasodilators such as Valsartan, a known Angiotensin Receptor Blocker or Enalapril, a known ACE Inhibitor, or calcium channel blockers used in the treatment of hypertension and congestive heart failure, for the prevention and treatment of alopecia.

ACE Inhibitors such as enalapril, benazepril, lisinopril, ramipril, or fosinopril or Angiotensin Receptor Blockers such as Valsartan, telmisartan, olmesartan, losartan, irbesartan, candesartan and azilsartan, when administered directly to the scalp, are very effective drugs for the prevention of alopecia.

Valsartan, or other Angiotensin Receptor Blockers, or Enalapril, or other ACE Inhibitors are drugs which previously may have been used in treatment of high blood pressure and or congestive heart failure, when applied in a pharmacological composition in an effective amount, directly to the scalp, by direct administration or subcutaneous injection, are effective drugs for the treatment of alopecia.

Angiotensin Receptor Blockers such as Valsartan, telmisartan, olmesartan, losartan, irbesartan, candesartan and azilsartan, when administered directly to the scalp, are very effective drugs for the prevention of alopecia.

Pharmacological composition as used herein is a pharmaceutical preparation according to the invention, composed but not limited to ACE Inhibitors, Calcium Channel Blockers, or Angiotensin Receptor Blockers and a suitable non-toxic pharmaceutical carrier.

Effective amount as used herein is an amount of the pharmaceutical composition of Calcium Channel Inhibitors, ACE Inhibitors, or Angiotensin Receptor Blockers that is effective for treating alopecia. An amount of Calcium Channel Inhibitors, ACE Inhibitors, or Angiotensin Receptor Blockers that is suitable for direct administration to the scalp or subcutaneous injection.

A method is provided of applying a pharmaceutical preparation in an effective amount of one or more vasodilators (e.g., calcium channel blockers, ACE inhibitors, angiotensin receptor blockers, nitrates, alpha blockers, beta blockers, hydralazine, and/or angiotensin receptor-neprilysin inhibitors), directly to the scalp or into the scalp by injection to treat or prevent alopecia.

The pharmacological preparation can comprise a calcium channel blocker. The calcium channel blocker can be in a suitable nontoxic pharmacological carrier.

The pharmacological preparation can comprise an ACE inhibitor. The ACE inhibitor can be in a suitable nontoxic pharmacological carrier.

The pharmacological preparation can comprise an angiotensin receptor blocker. The angiotensin receptor blocker can be in a suitable nontoxic pharmacological carrier.

An effective amount of calcium channel blocker for treatment or prevention of alopecia is administered. An amount of calcium channel blocker that is suitable for treatment by absorption through the scalp is administered.

An effective amount of ACE inhibitor for treatment or prevention of alopecia is administered. An amount of ACE inhibitor that is suitable for treatment by absorption through the scalp is administered.

An effective amount of angiotensin receptor for treatment or prevention of alopecia is administered. An amount of angiotensin receptor blocker that is suitable for by absorption through the scalp is administered.

Contact vasodilators (e.g., calcium channel blockers, ACE inhibitors, angiotensin receptor blockers, nitrates, alpha blockers, beta blockers, hydralazine, and/or angiotensin receptor-neprilysin inhibitors) are a new class of pharmaceutical medications that increase blood supply, which produces biological changes.

In the case of treatment or prevention of alopecia, these changes can include one or more of increasing the blood supply to the scalp.

Contact calcium channel blockers are a part of contact-vasodilators, a new class of medication. The use is provided of contact neo-vasodilators such as Nifedipine, a known calcium channel blocker used in the treatment of hypertension, for the prevention and treatment alopecia and alopecia related syndromes.

Nifedipine, Amlodipine, Felodipine, Isradipine, Nicardipine, Nisoldipine and Clevidipine are in a class of dihydropyridines calcium channel blockers. Verapamil and Diltiazem are non-dihydropyridines calcium channel blockers. When applied by contact these are very effective drugs for the treatment or prevention of alopecia and alopecia related syndromes.

Inhibitors of angiotensin converting enzyme (ACE) can be employed as vasodilators. Angiotensin II is a chemical produced by the body that primarily circulates in the blood. It causes the muscles surrounding blood vessels to contract, thereby narrowing the vessels. Angiotensin II is formed from angiotensin I in the blood by the enzyme angiotensin converting enzyme (ACE). Angiotensin I in the blood is itself formed from angiotensinogen, a protein produced by the liver and released into the blood. Angiotensin converting enzyme inhibitors (ACE inhibitors) are medications that slow (inhibit) the activity of the enzyme ACE, which decreases the production of angiotensin II. As a result, blood vessels enlarge or dilate. ACE inhibitors include, but are not limited to benazepril (Lotensin), captopril (Capoten), enalapril (Vasotec, Epaned, Lexxel), fosinopril (Monopril), lisinopril (Prinivil), moexipril (Univasc), perindopril (Aceon), quinapril (Accupril), ramipril (Altace), and trandolapril (Mavik).

Angiotensin II receptor blockers (ARBs) help relax the blood vessels. Angiotensin II receptor blockers block the action of angiotensin II, allowing blood vessels to dilate. Angiotensin receptor blockers include, but are not limited to: azilsartan (Edarbi), candesartan (Atacand), eprosartan, irbesartan (Avapro), losartan (Cozaar), olmesartan (Benicar), telmisartan (Micardis), and valsartan (Diovan).

Other vasodilators are known in the art. These include, but are not limited to nitrates (nitroglycerin, isosorbide mononitrate and isosorbide dinitrate), Alpha blockers (doxazosin (Cardura), prazosin (Minipress), terazosin), Beta blockers (Acebutolol (Sectral), Atenolol (Tenormin), Bisoprolol fumarate (Zebeta), Carvedilol (Coreg)—Combined alpha/beta blocker, Esmilol (Brevibloc), Labetalol (Trandate, Normodyne)—Combined alpha/beta blocker, Metoprolol tartrate (Lopressor) and metoprolol succinate (Toprol-XL), Nadolol (Corgard), Nebivolol (Bystolic), Penbutolol sulfate (Levatol), Propranolol (Inderal), Sotalol (Betapace), HCTZ and bisoprolol (Ziac) is a beta blocker plus diuretic), Hydralazine, and angiotensin receptor-neprilysin inhibitors (ARNi) (Entresto, sacubitril/valsartan).

### Conditions Amenable to Treatment or Prevention

Compositions and methods are provided for the prevention and treatment of alopecia and alopecia related syndromes, as appears herein is a syndrome comprised of any of the following: any visible or non visible hair loss on the scalp epidermis.

Application of vasodilators (e.g., calcium channel blockers, ACE inhibitors, angiotensin receptor blockers, nitrates, alpha blockers, beta blockers, hydralazine, and/or angiotensin receptor-neprilysin inhibitors), such as Nifedipine or other calcium channel blockers, which previously may have been used in the treatment of high blood pressure, in a pharmacological composition, in an effective amount, in a contact form, such as, but not limited to an oil, liquid preparation or suspension, for direct administration to the scalp or for administration by subcutaneous injection into the scalp, can be employed to treat or prevent the alopecia or the symptoms of alopecia.

Pharmacological compositions of the embodiments include but are not limited to one or more vasodilators (e.g., calcium channel blockers, ACE inhibitors, angiotensin receptor blockers, nitrates, alpha blockers, beta blockers, hydralazine, and/or angiotensin receptor-neprilysin inhibitors) and a suitable non toxic pharmaceutical carrier. The pharmaceutical composition in administered in an amount effective for treating alopecia or the symptoms of alopecia, e.g., an amount suitable for treatment by direct application to the scalp or by subcutaneous injection.

Alopecia, associated symptoms, and treatment thereof, or pharmaceutical compositions are described in the following references, each of which is incorporated by reference herein in its entirety and each of which is hereby made a part of this specification: Whiting DA. The structure of human hair follicle, Canfield Publishing, Fairfield, N.J. 2004; Childs J M, Sperling L C. Histopathology of scarring and nonscarring hair loss. Dermatol Clin 2013; 31:43; Schweizer J, Langbein L, Rogers M A, Winter H. Hair follicle-specific keratins and their diseases. Exp Cell Res 2007; 313:2010; Myung P, Ito M. Dissecting the bulge in hair regeneration. J Clin Invest 2012; 122:448; Mobini N, Tam S, Kamino H. Possible role of the bulge region in the pathogenesis of inflammatory scarring alopecia: lichen planopilaris as the prototype. J Cutan Pathol 2005; 32:675; Cotsarelis G, Botchkarev V. Biology of hair follicles. In: Fitzpatrick's Dermatology in General Medicine, 8th ed, Goldsmith L A, Katz S I, Gilchrest B A, et al (Eds), McGraw-Hill, New York 2012. Vol 1, p.960; Breitkopf T, Leung G, Yu M, et al. The basic science of hair biology: what are the causal mechanisms for the disordered hair follicle? Dermatol Clin 2013; 31:1; Rosenfield R L. Clinical practice. Hirsutism. N Engl J Med 2005; 353:2578; Messenger AG, Sinclair R. Follicular miniaturization in female pattern hair loss: clinicopathological correlations. Br J Dermatol 2006; 155:926; Price V H. Treatment of hair loss. N Engl J Med 1999; 341:964; Paus R, Cotsarelis G. The biology of hair follicles. N Engl J Med 1999; 341:491; Scheinfeld N S. A case of dissecting cellulitis and a review of the literature. Dermatol Online J 2003; 9:8; Powell J J, Dawber R P, Gatter K. Folliculitis decalvans including tufted folliculitis: clinical, histological and therapeutic findings. Br J Dermatol 1999; 140:328; Otberg N. Primary cicatricial alopecias. Dermatol Clin 2013; 31:155; Patel P, Werth V. Cutaneous lupus erythematosus: a review. Dermatol Clin 2002; 20:373; MacDonald A, Clark C, Holmes S. Frontal fibrosing alopecia: a review of 60 cases. J Am Acad Dermatol 2012; 67:955; RONCHESE F. Pseudopelade. Arch Dermatol 1960; 82:336; Kossard S, Collins A, McCrossin I. Necrotizing lymphocytic folliculitis: the early lesion of acne necrotica (varioliformis). J Am Acad Dermatol 1987; 16:1007; Whiting D A. Cicatricial alopecia: clinico-pathological findings and treatment. Clin Dermatol 2001; 19:211; Ito M, Liu Y, Yang Z, et al. Stem cells in the hair follicle bulge contribute to wound repair but not to homeostasis of the epidermis. Nat Med 2005; 11:1351; Lew B L, Shin M K, Sim W Y. Acute diffuse and total alopecia: A new subtype of alopecia areata with a favorable prognosis. J Am Acad Dermatol 2009; 60:85; Alkhalifah A, Alsantali A, Wang E, et al. Alopecia areata update: part I. Clinical picture, histopathology, and pathogenesis. J Am Acad Dermatol 2010; 62:177; Martinez-Mir A, Zlotogorski A, Gordon D, et al. Genomewide scan for linkage reveals evidence of several susceptibility loci for alopecia areata. Am J Hum Genet 2007; 80:316; Aksu Cerman A, Sarikaya Solak S, Kivanc Altunay I. Vitamin D deficiency in alopecia areata. Br J Dermatol 2014; 170:1299; Kubeyinje E P. Intralesional triamcinolone acetonide in alopecia areata amongst 62 Saudi Arabs. East Afr Med J 1994; 71:674; Chapel TA. The signs and symptoms of secondary syphilis. Sex Transm Dis 1980; 7:161; Khumalo N P, Ngwanya R M. Traction alopecia: 2% topical minoxidil shows promise. Report of two cases. J Eur Acad Dermatol Venereol 2007; 21:433; Hanly A J, Jorda M, Badiavas E, et al. Postoperative pressure-induced alopecia: report of a case and discussion of the role of apoptosis in non-scarring alopecia. J Cutan Pathol 1999; 26:357; Trancik R J, Spindler J R, Rose S, et al. Incidence of androgenetic alopecia in males 15 to 17 years of age. Poster presented at: 3rd Intercontinental Meeting of the Hair Research Societies Jun. 13-15, 2001, Tokyo. p.127; Paik J H, Yoon J B, Sim W Y, et al. The prevalence and types of androgenetic alopecia in Korean men and women. Br J Dermatol 2001; 145:95; Kaufman K D. Androgens and alopecia. Mol Cell Endocrinol 2002; 198:89; Imperato-McGinley J, Zhu Y S. Androgens and male physiology the syndrome of 5alpha-reductase-2 deficiency. Mol Cell Endocrinol 2002; 198:51; Sawaya M E. Purification of androgen receptors in human sebocytes and hair. J Invest Dermatol 1992; 98:92S; Norwood O T. Male pattern baldness: classification and incidence. South Med J 1975; 68:1359; Norwood O T. Incidence of female androgenetic alopecia (female pattern alopecia). Dermatol Surg 2001; 27:53; Mella J M, Perret M C, Manzotti M, et al. Efficacy and safety of finasteride therapy for androgenetic alopecia: a systematic review. Arch Dermatol 2010; 146:1141; Messenger A G, Rundegren J. Minoxidil: mechanisms of action on hair growth. Br J Dermatol 2004; 150:186; Messenger A G, Rundegren J. Minoxidil: mechanisms of action on hair growth. Br J Dermatol 2004; 150:186; Atanaskova Mesinkovska N, Bergfeld W F. Hair: what is new in diagnosis and management? Female pattern hair loss update: diagnosis and treatment. Dermatol Clin 2013; 31:119; Norwood O T. Incidence of female androgenetic alopecia (female pattern alopecia). Dermatol Surg 2001; 27:53; Birch M P, Messenger J F, Messenger A G. Hair density, hair diameter and the prevalence of female pattern hair loss. Br J Dermatol 2001; 144:297; Yip L, Rufaut N, Sinclair R. Role of genetics and sex steroid hormones in male androgenetic alopecia and female pattern hair loss: an update of what we now know. Australas J Dermatol 2011; 52:81; Messenger A G, Sinclair R. Follicular miniaturization in female pattern hair loss: clinicopathological correlations. Br J Dermatol 2006; 155:926; Arias-Santiago S, Gutiérrez-Salmerón M T, Buendía-Eisman A, et al. Hypertension and aldosterone levels in women with early-onset androgenetic alopecia. Br J Dermatol 2010; 162:786; Hirsso P, Rajala U, Laakso M, et al. Health-related quality of life and physical well-being among a 63-year-old cohort of women with androgenetic alopecia; a Finnish population-based study. Health Qual Life Outcomes 2005; 3:49; Su L H, Chen L S, Lin S C, Chen H H. Association of androgenetic alopecia with mortality from diabetes mellitus and heart disease. JAMA Dermatol 2013; 149:601; Trëb R M, Swiss Trichology Study Group. Finasteride treatment of patterned hair loss in normoandrogenic postmenopausal women. Dermatology 2004; 209:202; van Zuuren E J, Fedorowicz Z, Schoones J. Interventions for female pattern hair loss. Cochrane Database Syst Rev 2016; :CD007628; Olsen E A, Messenger A G, Shapiro J, et al. Evaluation and treatment of male and female pattern hair loss. J Am Acad Dermatol 2005; 52:301; Lachgar S, Charveron M, Gall Y, Bonafe J L. Minoxidil upregulates the expression of vascular endothelial growth factor in human hair dermal papilla cells. Br J Dermatol 1998; 138:407; Messenger A G, Rundegren J. Minoxidil: mechanisms of action on hair growth. Br J Dermatol 2004; 150:186; Lachgar S, Charveron M, Gall Y, Bonafe J L. Minoxidil upregulates the expression of vascular endothelial growth factor in human hair dermal papilla cells. Br J Dermatol 1998; 138:407; Li M, Marubayashi A, Nakaya Y, et al. Minoxidil-induced hair growth is mediated by adenosine in cultured dermal papilla cells: possible involvement of sulfonylurea receptor 2B as a target of minoxidil. J Invest Dermatol 2001; 117:1594; Messenger A G, Rundegren J. Minoxidil: mechanisms of action on hair growth. Br J Dermatol 2004; 150:186; Mella J M, Perret M C, Manzotti M, et al. Efficacy and safety of finasteride therapy for androgenetic alopecia: a systematic review. Arch Dermatol 2010; 146:1141; Ebner H, Müller E. Allergic contact dermatitis from minoxidil. Contact Dermatitis 1995; 32:316; Birch M P, Messenger J F, Messenger A G. Hair density, hair diameter and the prevalence of female pattern hair loss. Br J Dermatol 2001; 144:297; Casey J H. Chronic treatment regimens for hirsutism in women: effect on blood production rates of testosterone and on hair growth. Clin Endocrinol (Oxf) 1975; 4:313; Olsen E A, Messenger A G, Shapiro J, et al. Evaluation and treatment of male and female pattern hair loss. J Am Acad Dermatol 2005; 52:301; van Zuuren E J, Fedorowicz Z, Schoones J. Interventions for female pattern hair loss. Cochrane Database Syst Rev 2016; CD007628; Olsen E A, Dunlap F E, Funicella T, et al. A randomized clinical trial of 5% topical minoxidil versus 2% topical minoxidil and placebo in the treatment of androgenetic alopecia in men. J Am Acad Dermatol 2002; 47:377; De Villez R L. Topical minoxidil therapy in hereditary androgenetic alopecia. Arch Dermatol 1985; 121:197; Mella J M, Perret M C, Manzotti M, et al. Efficacy and safety of finasteride therapy for androgenetic alopecia: a systematic review. Arch Dermatol 2010; 146:1141; Arca E, Açikgöz G, Taşan H B, et al. An open, randomized, comparative study of oral finasteride and 5% topical minoxidil in male androgenetic alopecia. Dermatology 2004; 209:117; Saraswat A, Kumar B. Minoxidil vs finasteride in the treatment of men with androgenetic alopecia. Arch Dermatol 2003; 139:1219; Amory J K, Wang C, Swerdloff R S, et al. The effect of 5alpha-reductase inhibition with dutasteride and finasteride on semen parameters and serum hormones in healthy men. J Clin Endocrinol Metab 2007; 92:1659; Rahimi-Ardabili B, Pourandarjani R, Habibollahi P, Mualeki A. Finasteride induced depression: a prospective study. BMC Clin Pharmacol 2006; 6:7; D'Amico A V, Roehrborn C G. Effect of 1 mg/day finasteride on concentrations of serum prostate-specific antigen in men with androgenic alopecia: a randomised controlled trial. Lancet Oncol 2007; 8:21; Thompson I M, Goodman P J, Tangen C M, et al. The influence of finasteride on the development of prostate cancer. N Engl J Med 2003; 349:215; Godfraind, T., 2017. Discovery and development of calcium channel blockers. Frontiers in pharmacology, 8, p.286; Williams, B., Drug discovery in renin-angiotensin system intervention: past and future. Therapeutic Advances in Cardiovascular Disease, 2016 June; 10(3): 118-25; Shi L, Mao C, Xu Z, Zhang L. Angiotensin-converting enzymes and drug discovery in cardiovascular disease. Drug Discovery today, 2010, May 1;15(9-10):332-41.

Compositions including one or more vasodilators (e.g., calcium channel blockers, ACE inhibitors, angiotensin receptor blockers, nitrates, alpha blockers, beta blockers, hydralazine, and/or angiotensin receptor-neprilysin inhibitors), optionally in combination with conventional therapies (e.g., drug therapies or phototherapy), and associated methods for treatment of alopecia and related symptoms are provided.

Some embodiments relate to a pharmaceutical composition and method of treatment using the pharmaceutical composition, wherein the pharmaceutical composition comprises at least one calcium channel blocker, for example, a calcium channel blocker selected from the group consisting of amlodipine (Norvasc), diltiazem (Cardizem L A, Tiazac), felodipine (Plendil), isradipine (Dynacirc), nifedipine (Adalat, Procardia), nicardipine (Cardene), nimodipine (Nimotop), nisoldipine (Sular), verapamil (Covera-HS, Verelan P M, Calan), verapamil, diltiazem and nicardipine (Cardene IV). Some embodiments relate to a pharmaceutical composition and method of treatment using the pharmaceutical composition, wherein the pharmaceutical composition comprises at least one ACE inhibitors, for example at least one ACE inhibitor selected from the group consisting of benazepril (Lotensin), captopril (Capoten), enalapril (Vasotec, Epaned, Lexxel), fosinopril (Monopril), lisinopril (Prinivil), moexipril (Univasc), perindopril (Aceon), quinapril (Accupril), ramipril (Altace), and trandolapril (Mavik). Some embodiments relate to a pharmaceutical composition and method of treatment using the pharmaceutical composition, wherein the pharmaceutical composition comprises at least one angiotensin receptor blocker, for example at least one angiotensin receptor blocker selected from the group consisting of azilsartan (Edarbi), candesartan (Atacand), eprosartan, irbesartan (Avapro), losartan (Cozaar), olmesartan (Benicar), telmisartan (Micardis), and valsartan (Diovan). In certain embodiments, the pharmaceutical composition is in a form suitable for contact administration, e.g., to scalp tissue or epidermis or by subcutaneous administration by injection, however other routes of administration are also considered that involve contact of the vasodilator to the tissue to be treated.

The pharmaceutical compositions for treatment of alopecia can further comprise other pharmaceutically active ingredients. These can include drugs to control pain, for example, nonsteroidal anti-inflammatory drugs such as ibuprofen or naproxen sodium, topical anesthetics such as lidocaine, drugs to fight infections (e.g., antibiotic, antiviral, or antifungal agents). The treatment can be administered in conjunction with other therapies, e.g., the conventional therapies for alopecia as described elsewhere herein.

The use of topical vasodilators (e.g., calcium channel blockers, ACE inhibitors, angiotensin receptor blockers, nitrates, alpha blockers, beta blockers, hydralazine, and/or angiotensin receptor-neprilysin inhibitors) for treatment of alopecia or symptoms of hair loss is a new class of drugs. The new class may be used for alopecia or hair loss, or to enhance efficacy of conventional alopecia or hair loss drugs or non-drug treatments (e.g., phototherapy, photochemotherapy, corticosteroids, hair transplants, or the like).

In one method of the vasodilator may be applied directly to the tissue of the scalp, e.g., in a form of a cream or lotion.

In another embodiment, the vasodilator may be injected directly into the subcutaneous tissue to treat alopecia. The vasodilator may be applied even after the alopecia has been ameliorated to prevent recurrence of alopecia, or to maintain an improved condition.

### Definitions

The term “alcohol” as used herein is a broad term, and is to be given its ordinary and customary meaning to a person of ordinary skill in the art (and is not to be limited to a special or customized meaning), and refers without limitation to any compound as described herein incorporating one or more hydroxy groups, or being substituted by or functionalized to include one or more hydroxy groups.

The term “derivative” as used herein is a broad term, and is to be given its ordinary and customary meaning to a person of ordinary skill in the art (and is not to be limited to a special or customized meaning), and refers without limitation to any compound as described herein incorporating one or more derivative groups, or being substituted by or functionalized to include one or more derivative groups. Derivatives include but are not limited to esters, amides, anhydrides, acid halides, thioesters, and phosphates.

The term “hydrocarbon” as used herein is a broad term, and is to be given its ordinary and customary meaning to a person of ordinary skill in the art (and is not to be limited to a special or customized meaning), and refers without limitation to any moiety comprising only carbon and hydrogen atoms. A functionalized or substituted hydrocarbon moiety has one or more substituents as described elsewhere herein.

The term “lipid” as used herein is a broad term, and is to be given its ordinary and customary meaning to a person of ordinary skill in the art (and is not to be limited to a special or customized meaning), and refers without limitation to saturated and unsaturated oils and waxes, derivatives, amides, glycerides, fatty acids, fatty alcohols, sterol and sterol derivatives, tocopherols, carotenoids, among others.

The terms “pharmaceutically acceptable” as used herein is a broad term, and is to be given its ordinary and customary meaning to a person of ordinary skill in the art (and is not to be limited to a special or customized meaning), and refers without limitation to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable risk/benefit ratio.

The terms “pharmaceutically acceptable salts” and “a pharmaceutically acceptable salt thereof” as used herein are broad terms, and are to be given their ordinary and customary meaning to a person of ordinary skill in the art (and is not to be limited to a special or customized meaning), and refer without limitation to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as, for example, The Merck Index. Any suitable constituent can be selected to make a salt of the therapeutic agents discussed herein, provided that it is non-toxic and does not substantially interfere with the desired activity. In addition to salts, pharmaceutically acceptable precursors and derivatives of the compounds can be employed. Pharmaceutically acceptable amides, lower alkyl derivatives, and protected derivatives can also be suitable for use in compositions and methods of preferred embodiments. While it may be possible to administer the compounds of the preferred embodiments in the form of pharmaceutically acceptable salts, it is generally preferred to administer the compounds in neutral form.

The term “pharmaceutical composition” as used herein is a broad term, and is to be given its ordinary and customary meaning to a person of ordinary skill in the art (and is not to be limited to a special or customized meaning), and refers without limitation to a mixture of one or more pharmacologically active ingredients (e.g. vasodilators) disclosed herein with other chemical components, such as diluents or carriers. The pharmaceutical composition facilitates administration of the compound to an organism. Pharmaceutical compositions can also be obtained by reacting compounds with inorganic or organic acids or bases. Pharmaceutical compositions will generally be tailored to the specific intended route of administration.

As used herein, a “carrier” as used herein is a broad term, and is to be given its ordinary and customary meaning to a person of ordinary skill in the art (and is not to be limited to a special or customized meaning), and refers without limitation to a compound that facilitates the incorporation of a compound into cells or tissues. For example, without limitation, dimethyl sulfoxide (DMSO) is a commonly utilized carrier that facilitates the uptake of many organic compounds into cells or tissues of a subject. Water, saline solution, ethanol, and mineral oil are also carriers employed in certain pharmaceutical compositions.

As used herein, a “diluent” as used herein is a broad term, and is to be given its ordinary and customary meaning to a person of ordinary skill in the art (and is not to be limited to a special or customized meaning), and refers without limitation to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” as used herein is a broad term, and is to be given its ordinary and customary meaning to a person of ordinary skill in the art (and is not to be limited to a special or customized meaning), and refers without limitation to a substance that is added to a pharmaceutical composition to provide, without limitation, bulk, consistency, stability, binding ability, lubrication, disintegrating ability etc., to the composition. A “diluent” is a type of excipient.

As used herein, a “subject” as used herein is a broad term, and is to be given its ordinary and customary meaning to a person of ordinary skill in the art (and is not to be limited to a special or customized meaning), and refers without limitation to an animal that is the object of treatment, observation or experiment. “Animal” includes cold- and warm-blooded vertebrates and invertebrates such as fish, shellfish, reptiles, and, in particular, mammals. “Mammal” includes, without limitation, dolphins, mice, rats, rabbits, guinea pigs, dogs, cats, sheep, goats, cows, horses, primates, such as monkeys, chimpanzees, and apes, and, in particular, humans. In some embodiments, the subject is human.

As used herein, the terms “treating,” “treatment,” “therapeutic,” or “therapy” are broad terms, and are to be given their ordinary and customary meaning (and are not to be limited to a special or customized meaning) and, without limitation, do not necessarily mean total cure or abolition of the disease or condition. Any alleviation of any undesired markers, signs or symptoms of a disease or condition, to any extent, can be considered treatment and/or therapy. Furthermore, treatment may include acts that may worsen the patient's overall feeling of well-being or appearance.

The terms “therapeutically effective amount” and “effective amount” as used herein are broad terms, and are to be given its ordinary and customary meaning to a person of ordinary skill in the art (and are not to be limited to a special or customized meaning), and are used without limitation to indicate an amount of an active compound, or pharmaceutical agent, that elicits the biological or medicinal response indicated. For example, a therapeutically effective amount of compound can be the amount needed to prevent, alleviate or ameliorate markers or symptoms of a condition or prolong the survival of the subject being treated. This response may occur in a tissue, system, animal or human and includes alleviation of the signs or symptoms of the disease being treated. Determination of a therapeutically effective amount is well within the capability of those skilled in the art, in view of the disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

The term “solvents” as used herein is a broad term, and is to be given its ordinary and customary meaning to a person of ordinary skill in the art (and is not to be limited to a special or customized meaning), and refers without limitation to compounds with some characteristics of solvency for other compounds or means, that can be polar or nonpolar, linear or branched, cyclic or aliphatic, aromatic, naphthenic and that includes but is not limited to: alcohols, derivatives, diesters, ketones, acetates, terpenes, sulfoxides, glycols, paraffins, hydrocarbons, anhydrides, heterocyclics, among others.

It is to be understood that where compounds disclosed herein (e.g., calcium channel blockers, ACE inhibitors, angiotensin receptor blockers, nitrates, alpha blockers, beta blockers, hydralazine, and/or angiotensin receptor-neprilysin inhibitors) have unfilled valencies, then the valencies are to be filled with hydrogens or isotopes thereof, e.g., hydrogen-1 (protium) and hydrogen-2 (deuterium).

It is understood that the compounds described herein (e.g., calcium channel blockers, ACE inhibitors, angiotensin receptor blockers, nitrates, alpha blockers, beta blockers, hydralazine, and/or angiotensin receptor-neprilysin inhibitors) can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein may include crystalline forms (also known as polymorphs, which include the different crystal packing arrangements of the same elemental composition of a compound), amorphous phases, salts, solvates, and hydrates, e.g., of vasodilators. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein (e.g., vasodilators) may exist in unsolvated as well as solvated forms. In general, the solvated forms are considered equivalent to the unsolvated forms for the purposes of the compounds and methods provided herein.

Where a range of values is provided, it is understood that the upper and lower limit, and any intervening value between the upper and lower limit of the range is included.

Any percentages, ratios or other quantities referred to herein are on a weight basis, unless otherwise indicated.

### Pharmaceutical Compositions

The vasodilators (e.g., calcium channel blockers, ACE inhibitors, angiotensin receptor blockers, nitrates, alpha blockers, beta blockers, hydralazine, and/or angiotensin receptor-neprilysin inhibitors) can be prepared by any suitable method known to those in the art. For representative methods, see, for example, Francis A. Carey et al., Advanced Organic Chemistry: Part B: Reaction and Synthesis (5^{th} Ed. 2005).

Formulations including a vasodilator (e.g., a calcium channel blocker, ACE inhibitor and/or angiotensin receptor blocker) and at least one excipient are provided. It is generally preferred to administer the compounds of the embodiments in topical formulations; however, other routes of administration are also contemplated.

The pharmaceutical compositions described herein can be administered by themselves to a subject, or in compositions where they are mixed with other active agents, as in combination therapy, or with carriers, diluents, excipients or combinations thereof. Formulation is dependent upon the route of administration chosen. Techniques for formulation and administration of the compounds described herein are known to those skilled in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The pharmaceutical compositions disclosed herein may be manufactured by a process that is itself known, e.g., by means of conventional mixing, dissolving, granulating, dragee-making, levigating, emulsifying, encapsulating, entrapping, tableting, or extracting processes. Many of the vasodilator (e.g., a calcium channel blocker, ACE inhibitor and/or angiotensin receptor blocker) used in the pharmaceutical combinations disclosed herein may be provided as salts with pharmaceutically acceptable counterions.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections. Contemplated herein is any combination of the forgoing, or other methods as would be known to one of ordinary skill in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The compositions described herein are suitable for use in treatment or prevention of alopecia or associated symptoms. The compositions are suitable for use in any patient where treatment or prevention of alopecia is desirable.

The vasodilator (e.g., a calcium channel blocker, ACE inhibitor and/or angiotensin receptor blocker) can be employed in various types of formulations. Topical formulations including one or more vasodilators in combination with at least one excipient are provided. Excipients can include a nonaqueous or aqueous carrier, and one or more agents selected from moisturizing agents, pH adjusting agents, deodorants, fragrances, chelating agents, preservatives, emulsifiers, thickeners, solubilizing agents, penetration enhancers, anti-irritants, colorants, surfactants, beneficial agents, pharmaceutical agents, and other components as known in the art for use in connection with topical formulations for application to skin or epidermis. The formulation can be provided as an aqueous formulation, or in an anhydrous formulation which may prevent water-based irritant contact dermatitis or stinging sensation upon application. In another embodiment, the composition is formulated such that preservatives need not be employed (e.g., a preservative-free formulation) so as to avoid skin irritation associated with certain preservatives.

To facilitate application, the composition may be provided as an ointment, an oil, a lotion, a paste, a powder, a gel, or a cream. The composition may also include additional ingredients such as a protective agent, an emollient, a humectant, an antibiotic agent, an antifungal agent, an antiviral agent, an antiprotozoal agent, an anesthetic agent, a steroidal anti-inflammatory agent, a non-steroidal anti-inflammatory agent, an antipruritic agent, an antioxidant agent, an anti-histamine agent, a vitamin or vitamin complex, a hormone, an anti-skin atrophy agent, and combinations thereof. In a further embodiment, the composition may avoid animal or cellular-based materials to avoid irritation. The composition can be applied directly to the tissue of scalp or epidermis.

Methods of using vasodilator formulations are provided. The compositions may be applied topically, but may also be applied to the tissue to be treated via subcutaneous injection.

Some embodiments include administering vasodilator (e.g., a calcium channel blocker, ACE inhibitor and/or angiotensin receptor blocker) compositions provided herein in topical formulations; however, other routes of administration are also contemplated (e.g., subcutaneous or the like). Contemplated routes of administration include but are not limited to topical and subcutaneous. Suitable liquid forms include suspensions, emulsions, solutions, and the like. Unit dosage forms can also be provided, e.g., individual packets with a premeasured amount of the formulation, configured for administration to the tissue on a predetermined schedule (e.g., daily, weekly, etc.). Unit dosage forms configured for administration twice a day can be employed; however, in certain embodiments it can be desirable to configure the unit dosage form for administration once a day, four times a day, or more, or once every other day, every three days, weekly, or less, or on an as-needed basis.

In some embodiments, the topical and subcutaneous formulations typically comprise from about 0.001 wt. % or less to about 50 wt. % or more of active ingredient, such as the vasodilator (e.g., a calcium channel blocker, ACE inhibitor and/or angiotensin receptor blocker), preferably from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %.

Compositions and formulations for topical administration to the tissue of the scalp or epidermis can include gels, drops, sprays, liquids, and aerosols. Conventional pharmaceutical carriers, aqueous or oily bases, thickeners and the like may be employed. Such formulations are typically provided in a squeeze bottle. A liquid or gel can also be placed using an applicator, e.g., a wand, a sponge, a syringe, or other suitable method.

A topical formulation can be provided in a form of a carrier containing the vasodilator, e.g., 50 ppm or less to 1000, 5000, 10000, 50000, 100000, 500000 ppm or more of the vasodilator. The topical formulation can contain from 0.01 wt. % or less (e.g., 0.001 wt. %) to 10 wt. % or more, e.g., 0.01 wt. % to 0.02 wt. %, 0.03 wt. %, 0.04 wt. %, 0.05 wt. %, 0.1 wt. %, 1 wt. % to 5 wt. % or 10 wt. % or 20 wt. % of the vasodilator. The amount of vasodilator in the base can be adjusted up or down.

Liquids and gels containing the vasodilator, optionally with other components as described herein, can be prepared using techniques as are known in the art for preparing topical compositions. See, e.g., Handbook of Cosmetic Science and Technology, Fourth Edition, edited by André O. Barel, Marc Paye, Howard I. Maibach, CRC Press, 2014, the contents of which is hereby incorporated by reference in its entirety. Various formulations are possible.

For liquid formulations (e.g., gel or lotion forms), a silicone, e.g., a cyclosiloxane or linear silicone (e.g., silicone elastomer), can be employed as a carrier. One type of suitable carrier is a dimethicone crosspolymer gel, e.g., dimethicone crosspolymer in cyclopentasiloxane. Other suitable dimethicone crosspolymers include cyclopentasiloxane, dimethicone/vinyldimethicone crosspolymer; dimethicone, dimethicone/vinyl dimethicone crosspolymer; and isodecane dimethicone/vinyl dimethicone crosspolymer.

Typically, the carrier is present in an amount of from about 80 wt. % to about 95 wt. %, or 82 wt. % to 92 wt. %, e.g., in a topical formulation for application to the epidermis or scalp.

Penetration enhancers can be employed to enhance penetration of the vasodilator into tissue. Typical amounts when employed in topical formulations are from 1% by weight to 4% by weight. Typical amounts for anti-irritation agents when employed in topical formulations are from 1% by weight to 4% by weight. Typical amounts for anti-inflammatory agents when employed in topical formulations are from 1% by weight to 4% by weight. Typical amounts for anti-inflammatory agents when employed in topical formulations are from 0.1% by weight to 2% by weight.

In some embodiments, the vasodilator can be in admixture with a suitable carrier, diluent, or excipient, and can contain auxiliary substances such as wetting or emulsifying agents, pH buffering agents, gelling or viscosity enhancing additives, preservatives, scenting agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulations include, without limitation, monoglycerides, diglycerides, sulfatides, lysolecithin, phospholipids, saponin, bile acids, and the like. The presence of such additional components can influence the physical state, solubility, stability, rate of release, rate of clearance, and penetration of active ingredients.

The compositions for topical administration to the tissue of the scalp or epidermis comprise the vasodilator as described herein and a vehicle acceptable for contact with the tissue. The vehicle may be aqueous or nonaqueous. The vehicle used in the topical composition may be in the form of a gel, an ointment, a liquid, a cream, or an emulsion. If the vehicle is an emulsion, the emulsion may have a continuous aqueous phase and a discontinuous nonaqueous or oil phase (oil-in-water emulsion), or a continuous nonaqueous or oil phase and a discontinuous aqueous phase (water-in-oil emulsion). When administered topically in liquid or gel form, a liquid carrier such as water, petroleum, oils of animal or plant origin such as peanut oil, mineral oil, soybean oil, or sesame oil, or synthetic oils can be added to the active ingredient(s). Physiological saline solution, dextrose, or other saccharide solution, or glycols such as ethylene glycol, propylene glycol, or polyethylene glycol are also suitable liquid carriers. The pharmaceutical compositions can also be in the form of oil-in-water emulsions. The oily phase can be a vegetable oil, such as olive or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions can also contain coloring and scenting agents.

In certain embodiments, a silicone elastomer (e.g., dimethicone crosspolymer) is employed to increase delivery and penetration of the vasodilator into the scalp or epidermis.

The pharmaceutical excipients used in the topical preparations of the vasodilator compositions may be selected from the group consisting of solvents, emollients and/or emulsifiers, oil bases, preservatives, antioxidants, tonicity adjusters, penetration enhancers and solubilizers, chelating agents, buffering agents, surfactants, one or more polymers, and combinations thereof.

Suitable solvents for an aqueous or hydrophilic topical formulation include water; ethyl alcohol; isopropyl alcohol; mixtures of water and ethyl and/or isopropyl alcohols; glycerin; ethylene, propylene or butylene glycols; DMSO; and mixtures thereof. Suitable solvents for hydrophobic topical formulations include mineral oils, vegetable oils, and silicone oils. If desired, the vasodilator compositions as described herein may be dissolved or dispersed in a hydrophobic oil phase, and the oil phase may then be emulsified in an aqueous phase comprising water, alone or in combination with lower alcohols, glycerin, and/or glycols. In certain embodiments water is present, but at amounts below the threshold at which a stinging sensation when applied to damaged skin may result. Osmotic shock or osmotic stress is a sudden change in the solute concentration around a cell, causing a rapid change in the movement of water across its cell membrane. Under conditions of high concentrations of either salts, substrates or any solute in the supernatant, water is drawn out of the cells through osmosis. This also inhibits the transport of substrates and cofactors into the cell thus “shocking” the cell. Alternatively, at low concentrations of solutes, water enters the cell in large amounts, causing it to swell and either burst or undergo apoptosis. Certain of the formulations as described herein can be advantageously employed where it is desirable to minimize osmotic shock.

Viscosity of the compositions can be maintained at the selected level using a pharmaceutically acceptable thickening agent. Suitable viscosity enhancers or thickeners which may be used to prepare a viscous gel or cream with an aqueous base include sodium polyacrylate, xanthan gum, polyvinyl pyrrolidone, acrylic acid polymer, carragenans, hydroxyethyl cellulose, hydroxypropyl cellulose, methyl cellulose, ethyl cellulose, propyl cellulose, hydroxypropyl methyl cellulose, polyethoxylated polyacrylamides, polyethoxylated acrylates, and polyethoxylated alkane thiols. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the thickening agent selected. An amount is preferably used that will achieve the selected viscosity. Viscous compositions are normally prepared from solutions by the addition of such thickening agents, or by employing a base that has an acceptable level of viscosity.

Suitable emollients include hydrocarbon oils and waxes such as mineral oil, petrolatum, paraffin, ceresin, ozokerite, microcrystalline wax, polyethylene, squalene, perhydrosqualene, silicone oils, triglyceride esters, acetoglyceride esters, such as acetylated monoglycerides; ethoxylated glycerides, such as ethoxylated glyceryl monostearate; alkyl esters of fatty acids or dicarboxylic acids.

Suitable silicone oils for use as emollients include dimethyl polysiloxanes, methyl(phenyl) polysiloxanes, and water-soluble and alcohol-soluble silicone glycol copolymers. Suitable triglyceride esters for use as emollients include vegetable and animal fats and oils including castor oil, safflower oil, cotton seed oil, corn oil, olive oil, cod liver oil, almond oil, avocado oil, palm oil, sesame oil, and soybean oil.

Suitable esters of carboxylic acids or diacids for use as emollients include methyl, isopropyl, and butyl esters of fatty acids. Specific examples of alkyl esters including hexyl laurate, isohexyl laurate, iso-hexyl palmitate, isopropyl palmitate, decyl oleate, isodecyl oleate, hexadecyl stearate, decyl stearate, isopropyl isostearate, dilauryl lactate, myristyl lactate, and cetyl lactate; and alkenyl esters of fatty acids such as oleyl myristate, oleyl stearate, and oleyl oleate. Specific examples of alkyl esters of diacids include diisopropyl adipate, diisohexyl adipate, bis(hexyldecyl) adipate, and diisopropyl sebacate.

Other suitable classes of emollients or emulsifiers which may be used in the topical formulations include fatty acids, fatty alcohols, fatty alcohol ethers, ethoxylated fatty alcohols, fatty acid esters of ethoxylated fatty alcohols, and waxes.

Specific examples of fatty acids for use as emollients include pelargonic, lauric, myristic, palmitic, stearic, isostearic, hydroxystearic, oleic, linoleic, ricinoleic, arachidic, behenic, and erucic acids. Specific examples of fatty alcohols for use as emollients include lauryl, myristyl, cetyl, hexadecyl, stearyl, isostearyl, hydroxystearyl, oleyl, ricinoleyl, behenyl, and erucyl alcohols, as well as 2-octyl dodecanol.

Specific examples of waxes suitable for use as emollients include lanolin and derivatives thereof including lanolin oil, lanolin wax, lanolin alcohols, lanolin fatty acids, isopropyl lanolate, ethoxylated lanolin, ethoxylated lanolin alcohols, ethoxolated cholesterol, propoxylated lanolin alcohols, acetylated lanolin, acetylated lanolin alcohols, lanolin alcohols linoleate, lanolin alcohols recinoleate, acetate of lanolin alcohols recinoleate, acetate of lanolin alcohols recinoleate, acetate of ethoxylated alcohols esters, hydrogenolysates of lanolin, hydrogenated lanolin, ethoxylated hydrogenated lanolin, ethoxylated sorbitol lanolin, and liquid and semisolid lanolin. Also usable as waxes include hydrocarbon waxes, ester waxes, and amide waxes. Useful waxes include wax esters such as beeswax, spermaceti, myristyl myristate and stearyl stearate; beeswax derivatives, e.g., polyoxyethylene sorbitol beeswax; and vegetable waxes including carnauba and candelilla waxes.

Polyhydric alcohols and polyether derivatives may be used as solvents and/or surfactants in the topical formulations. Suitable polyhydric alcohols and polyethers include propylene glycol, dipropylene glycol, polypropylene glycols 2000 and 4000, poly(oxyethylene-co-oxypropylene) glycols, glycerol, sorbitol, ethoxylated sorbitol, hydroxypropylsorbitol, polyethylene glycols 200-6000, methoxy polyethylene glycols 350, 550, 750, 2000 and 5000, poly[ethylene oxide] homopolymers (100,000-5,000,000), polyalkylene glycols and derivatives, hexylene glycol, 2-methyl-2,4-pentanediol, 1,3-butylene glycol, 1,2,6-hexanetriol, 2-ethyl-1,3-hexanediol, vicinal glycols having 15 to 18 carbon atoms, and polyoxypropylene derivatives of trimethylolpropane.

Polyhydric alcohol esters may be used as emulsifiers or emollients. Suitable polyhydric alcohol esters include ethylene glycol mono- and di-fatty acid esters, diethylene glycol mono- and di-fatty acid esters, polyethylene glycol (200-6000) mono- and di-fatty acid esters, propylene glycol mono- and di-fatty esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Suitable emulsifiers for use in topical formulations include anionic, cationic, nonionic, and zwitterionic surfactants. Preferred ionic emulsifiers include phospholipids, such as lecithin and derivatives.

Lecithin and other phospholipids may be used to prepare liposomes containing the vasodilators as described herein. Formation of lipid vesicles occurs when phospholipids such as lecithin are placed in water and consequently form one bilayer or a series of bilayers, each separated by water molecules, once enough energy is supplied. Liposomes can be created by sonicating phospholipids in water. Low shear rates create multilamellar liposomes. Continued high-shear sonication tends to form smaller unilamellar liposomes. Hydrophobic chemicals can be dissolved into the phospholipid bilayer membrane. The lipid bilayers of the liposomes deliver the vasodilators as described herein.

The topical formulation may contain micelles, or an aggregate of surfactant molecules dispersed in an aqueous solution. Micelles may be prepared by dispersing an oil solvent in an aqueous solution comprising a surfactant, where the surfactant concentration exceeds the critical micelle concentration. The resulting formulation contains micelles, i.e., spherical oil droplets surrounded by a membrane of polar surfactant molecules, dispersed in the aqueous solvent.

Sterols including, for example, cholesterol and cholesterol fatty acid esters; amides such as fatty acid amides, ethoxylated fatty acid amides, and fatty acid alkanolamides may also be used as emollients and/or penetration enhancers.

A pharmaceutically acceptable preservative can be employed to increase the shelf life of the composition. Other suitable preservatives and/or antioxidants for use in topical formulations include benzalkonium chloride, benzyl alcohol, phenol, urea, parabens, butylated hydroxytoluene (BHT), butylated hydroxyanisole (BHA), tocopherol, thimerosal, chlorobutanol, or the like, and mixtures thereof, can be employed. If a preservative, such as an antioxidant, is employed, the concentration is typically from about 0.02% to about 2% based on the total weight of the composition, although larger or smaller amounts can be desirable depending upon the agent selected. Reducing agents, as described herein, can be advantageously used to maintain good shelf life of the formulation. It is generally observed that the anhydrous formulations of the embodiments exhibit satisfactory stability, such that a preservative can be omitted from the formulation.

Suitable chelating agents for use in topical formulations include ethylene diamine tetraacetic acid, alkali metal salts thereof alkaline earth metal salts thereof, ammonium salts thereof, and tetraalkyl ammonium salts thereof.

The carrier preferably has a pH of between about 4.0 and 10.0, more preferably between about 6.8 and about 7.8. The pH may be controlled using buffer solutions or other pH modifying agents. Suitable pH modifying agents include phosphoric acid and/or phosphate salts, citric acid and/or citrate salts, hydroxide salts (i.e., calcium hydroxide, sodium hydroxide, potassium hydroxide) and amines, such as triethanolamine. Suitable buffer solutions include a buffer comprising a solution of monopotassium phosphate and dipotassium phosphate, maintaining a pH of between 5.8 and 8; and a buffer comprising a solution of monosodium phosphate and disodium phosphate, maintaining a pH of between 6 and 7.5. Other buffers include citric acid/sodium citrate, and dibasic sodium phosphate/citric acid. The vasodilator compositions of the embodiments are preferably isotonic with the blood or other body fluid of the recipient. The isotonicity of the compositions can be attained using sodium tartrate, propylene glycol or other inorganic or organic solutes. Sodium chloride is particularly preferred. Buffering agents can be employed, such as acetic acid and salts, citric acid and salts, boric acid and salts, and phosphoric acid and salts. It can be desirable to include a reducing agent in the formulation, such as vitamin C, vitamin E, or other reducing agents as are known in the pharmaceutical arts.

Surfactants can also be employed as excipients, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

When the vasodilator formulations of the embodiments are administered by subcutaneous injection, it is preferably in the form of a pyrogen-free, parenterally acceptable aqueous solution or oleaginous suspension, emulsion or solution. Suspensions can be formulated according to methods well known in the art using suitable dispersing or wetting agents and suspending agents. The preparation of acceptable aqueous or nonaqueous solutions with suitable properties, e.g., pH, isotonicity, stability, and the like, is within the skill in the art. For example, an isotonic vehicle such as 1,3-butanediol, water, isotonic sodium chloride solution, Ringer's solution, dextrose solution, dextrose and sodium chloride solution, lactated Ringer's solution, or other vehicles as are known in the art can be employed, or a fixed oil can be employed conventionally as a solvent or suspending medium, e.g., synthetic mono or diglycerides, fatty acids, or the like. The vasodilator formulations can also contain stabilizers, preservatives, buffers, antioxidants, or other additives known to those of skill in the art.

In certain embodiments, it can be advantageous to include additional agents having pharmacological activity. Anti-infective agents include, but are not limited to, anthelmintic (mebendazole), antibiotics including aminoglycosides (gentamicin, neomycin, tobramycin), antifungal antibiotics (amphotericin b, fluconazole, griseofulvin, itraconazole, ketoconazole, nystatin, micatin, tolnaftate), cephalosporins (cefaclor, cefazolin, cefotaxime, ceftazidime, ceftriaxone, cefuroxime, cephalexin), beta-lactam antibiotics (cefotetan, meropenem), chloramphenicol, macrolides (azithromycin, clarithromycin, erythromycin), penicillins (penicillin G sodium salt, amoxicillin, ampicillin, dicloxacillin, nafcillin, piperacillin, ticarcillin), tetracyclines (doxycycline, minocycline, tetracycline), bacitracin, clindamycin, colistimethate sodium, polymyxin b sulfate, vancomycin, antivirals including acyclovir, amantadine, didanosine, efavirenz, foscarnet, ganciclovir, indinavir, lamivudine, nelfinavir, ritonavir, saquinavir, stavudine, valacyclovir, valganciclovir, zidovudine, quinolones (ciprofloxacin, levofloxacin), sulfonamides (sulfadiazine, sulfisoxazole), sulfones (dapsone), furazolidone, metronidazole, pentamidine, sulfanilamidum crystallinum, gatifloxacin, and sulfamethoxazole/trimethoprim. Anesthetics can include, but are not limited to, ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and phenazopyridine. Anti-inflammatory agents include but are not limited to, nonsteroidal anti-inflammatory drugs (NSAIDs) such as aspirin, celecoxib, choline magnesium trisalicylate, diclofenac potassium, diclofenac sodium, diflunisal, etodolac, fenoprofen, flurbiprofen, ibuprofen, indomethacin, ketoprofen, ketorolac, melenamic acid, nabumetone, naproxen, naproxen sodium, oxaprozin, piroxicam, rofecoxib, salsalate, sulindac, and tolmetin; and corticosteroids such as cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinonide, betamethasone dipropionate, betamethasone valerate, desonide, desoximetasone, fluocinolone, triamcinolone, clobetasol propionate, and dexamethasone.

### Kits for Administration of Compositions

Some embodiments of the methods and compositions provided herein include kits comprising vasodilators provided herein. In some embodiments, kits can be provided to an administering physician, other health care professional, a patient, or a caregiver. In some embodiments, a kit comprises a container which contains the vasodilator(s) in a suitable topical formulation, and instructions for administering the composition to a subject. The kit can optionally also contain one or more additional therapeutic or other agents. For example, a kit containing a vasodilator blocker in topical form can be provided along with other agents such as topical antibiotics or topical anesthetics. The kit may contain the vasodilator in bulk form, or can contain separate doses of the vasodilator for serial or sequential administration. The kit can optionally contain one or more diagnostic tools, administration tools, and/or instructions for use, e.g., syringes for subcutaneous injection. The kit can contain suitable delivery devices, such as, patches, syringes, pump dispensers, wands, single dose packets, and the like, along with instructions for administering the vasodilator compositions and any other therapeutic or beneficial agents. The kit can optionally contain instructions for storage, reconstitution (if applicable), and administration of any or all therapeutic or beneficial agents included. The kits can include a plurality of containers reflecting the number of administrations to be given to a subject, or the different products to be administered to the subject.

The topical formulation for administration to tissue of the scalp or epidermis, in addition to the vasodilator, can contain other ingredients.

While topical administration of the vasodilator disclosed herein can advantageously be employed, in certain embodiments other routes of administration are also contemplated, such as subcutaneous injection.

The vasodilator compositions described herein can be administered by themselves to a subject, or in compositions where they are mixed with other active agents, as in combination therapy, or with carriers, diluents, excipients or combinations thereof. Formulation is dependent upon the route of administration chosen. Techniques for formulation and administration of the compounds described herein are known to those skilled in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The vasodilator compositions disclosed herein may be manufactured into administrable forms by a process that is itself known, e.g., by means of conventional mixing, dissolving, granulating, dragee-making, levigating, emulsifying, encapsulating, entrapping, tableting, or extracting processes.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections. Contemplated herein is any combination of the forgoing, or other methods as would be known to one of ordinary skill in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

In practice, the vasodilator may be combined as the active ingredient in intimate admixture with a pharmaceutical carrier according to conventional pharmaceutical compounding techniques. The excipients are preferably minimized so as to ensure administration of an appropriate amount of vasodilator in a compact format. The carrier can take a wide variety of forms depending on the form of preparation desired for administration. Thus, the vasodilator compositions provided herein can be presented as discrete units suitable for administration each containing a predetermined amount of the active ingredient. Further, the vasodilator compositions can be presented as an oil, as a solution, as a suspension in an aqueous liquid, as a non-aqueous liquid, as an oil-in-water emulsion, or as a water-in-oil liquid emulsion, similar to the topical formulations described elsewhere herein, but using components suitable for human contact or consumption. In addition to the common dosage forms set out above, the vasodilator compositions provided herein can also be administered by controlled release and/or delivery devices. The vasodilator compositions can be prepared by any of the methods of pharmacy. In general, such methods include a step of bringing into association the active ingredient with the carrier that constitutes one or more necessary ingredients. In general, the vasodilator compositions are prepared by uniformly and intimately admixing the vasodilator ingredient(s) with liquid carriers or finely divided solid carriers or both. The product can then be conveniently shaped into the desired presentation.

A vasodilator formulation may also be administered in a local manner, for example, via injection of the vasodilator composition directly into a target area, e.g., in a depot or sustained release formulation subcutaneously. Furthermore, a targeted drug delivery system for the vasodilator may be used, for example, in a liposome coated with a tissue specific antibody.

The vasodilator compositions may contain the vasodilator in an amount effective for the desired therapeutic effect. In some embodiments, the vasodilator compositions are in a unit dosage form and comprise from about 0.1 mg or less to about 5000 mg or more of vasodilator per unit dosage form. In further embodiments, the vasodilator compositions comprise from about 1 to about 500 mg per unit dosage form or from about 500 to 5000 mg per unit dosage form of vasodilator. Such amounts can be selected depending upon the vasodilator employed. Such dosage forms may be solid, semisolid, liquid, an emulsion, or adapted for delivery via aerosol or the like.

The carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, lower alcohols, and water. Examples of gaseous carriers include carbon dioxide and nitrogen.

Vasodilator compositions provided herein can be prepared as solutions or suspensions of the vasodilator in water or nonaqueous liquids. A suitable surfactant can be included such as, for example, hydroxypropylcellulose. Dispersions can also be prepared in glycerol, liquid polyethylene glycols, and mixtures thereof in oils. Further, a preservative can be included to, for example, prevent the detrimental growth of microorganisms.

Vasodilator compositions provided herein suitable for injectable use include sterile aqueous solutions or dispersions. Furthermore, the vasodilator compositions can be in the form of sterile powders for the extemporaneous preparation of such sterile injectable solutions or dispersions. The vasodilator compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

In addition to the aforementioned carrier ingredients, the vasodilator formulations described above can include, as appropriate, one or more additional carrier ingredients such as diluents, buffers, binders, surface-active agents, thickeners, lubricants, preservatives (including anti-oxidants) and the like. Furthermore, other adjuvants can be included to render the formulation isotonic with the blood or other bodily fluids of the intended recipient. Vasodilator compositions can also be prepared in powder or liquid concentrate form for dilution.

Contemplated herein are vasodilator compositions including one or more vasodilators as described herein in combination with at least one additional active agent, e.g., an antibiotic. The vasodilator and the at least one additional active agent(s) may be present in a single formulation or in multiple formulations provided together, or may be unformulated. In some embodiments, the vasodilator can be administered with one or more additional agents together in a single composition. For example, the vasodilator can be administered in one composition, and at least one of the additional agents can be administered in a second composition. In a further embodiment, the vasodilator and the at least one additional active agent(s) are co-packaged in a kit. For example, a drug manufacturer, a drug reseller, a physician, a compounding shop, or a pharmacist can provide a kit comprising the vasodilator in combination with another product or component for delivery to a patient. Such additional components can include anti-infective agents, anti-inflammatory agents, anesthetics, or the like.

Some embodiments described herein relate to compositions of vasodilator, which can include a therapeutically effective amount of the vasodilator described herein and a pharmaceutically acceptable carrier, diluent, excipient or combination thereof. The vasodilator composition can include the vasodilator in an amount for example, >1%, ≥2%, ≥3%, ≥4%, ≥5%, ≥6%, ≥7%, ≥8%, ≥9%, ≥10%, ≥20%, ≥30%, ≥40%, ≥50%, ≥60%, ≥70%, ≥80%, >90%, ≥95%, or ≥98% of the composition.

### EXAMPLES

### Example 1

A male patient is diagnosed with alopecia. A composition comprising the calcium channel blocker nifedipine is directly applied to the scalp on one side of the head while the other side is left untreated. The treated side is observed to have reduced symptoms of alopecia than the untreated side.

### Example 2

A male patient is diagnosed with alopecia. A composition comprising the ACE inhibitor enalapril is directly applied to the scalp on one side of the head while the other side is left untreated. The treated side is observed to have reduced symptoms of alopecia than the untreated side.

### Example 3

A male patient is diagnosed with alopecia. A composition comprising the angiotensin receptor blocker losartan is directly applied to the scalp on one side of the head while the other side is left untreated. The treated side is observed to have reduced symptoms of alopecia than the untreated side.

### Example 4

A female patient is diagnosed with alopecia. A composition comprising the calcium channel blocker nifedipine is directly applied to the scalp on one side of the head while the other side is left untreated. The treated side is observed to have reduced symptoms of alopecia than the untreated side.

### Example 5

A female patient is diagnosed with alopecia. A composition comprising the ACE inhibitor enalapril is directly applied to the scalp on one side of the head while the other side is left untreated. The treated side is observed to have reduced symptoms of alopecia than the untreated side.

### Example 6

A female patient is diagnosed with alopecia. A composition comprising the angiotensin receptor blocker losartan is directly applied to the scalp on one side of the head while the other side is left untreated. The treated side is observed to have reduced symptoms of alopecia than the untreated side.

### Example 7

A male patient is diagnosed with alopecia. A composition comprising the calcium channel blocker nifedipine is directly injected into the scalp on one side of the head while the side is left untreated. The treated side is observed to have reduced symptoms of alopecia than the untreated side.

### Example 8

A male patient is diagnosed with alopecia. A composition comprising the ACE inhibitor enalapril is directly injected into the scalp on one side of the head while the side is left untreated. The treated side is observed to have reduced symptoms of alopecia than the untreated side.

### Example 9

A male patient is diagnosed with alopecia. A composition comprising the angiotensin receptor blocker losartan is directly injected into the scalp on one side of the head while the side is left untreated. The treated side is observed to have reduced symptoms of alopecia than the untreated side.

### Example 10

A female patient is diagnosed with alopecia. A composition comprising the calcium channel blocker nifedipine is directly injected into the scalp on one side of the head while the side is left untreated. The treated side is observed to have reduced symptoms of alopecia than the untreated side.

### Example 11

A female patient is diagnosed with alopecia. A composition comprising the ACE inhibitor enalapril is directly injected into the scalp on one side of the head while the side is left untreated. The treated side is observed to have reduced symptoms of alopecia than the untreated side.

### Example 12

A female patient is diagnosed with alopecia. A composition comprising the angiotensin receptor blocker losartan is directly injected into the scalp on one side of the head while the side is left untreated. The treated side is observed to have reduced symptoms of alopecia than the untreated side.

### Exemplary Pharmaceutical Compositions and Methods

Pharmaceutical Composition 1: A pharmaceutical composition for the treatment or prophylaxis of alopecia, comprising: at least one vasodilator; and at least one pharmaceutical excipient.

Pharmaceutical Composition 2: Pharmaceutical Composition 1, for the treatment or prophylaxis of alopecia in males.

Pharmaceutical Composition 3: Pharmaceutical Composition 1, for the treatment or prophylaxis of alopecia in females.

Pharmaceutical Composition 4: Any One of Pharmaceutical Compositions 1 through 3, in a form adapted for direct administration or subcutaneous injection to the scalp or epidermis.

Pharmaceutical Composition 5: Pharmaceutical Composition 4, wherein the form is selected from the group consisting of an oil, a liquid and a suspension for direct application on the scalp or epidermis.

Pharmaceutical Composition 6: Any One of Pharmaceutical Compositions 1 through 3, formulated as a liquid or a suspension of the at least one vasodilator, wherein the vasodilator is a contact vasodilator.

Pharmaceutical Composition 7: Any One of Pharmaceutical Compositions 1 through 6, wherein the vasodilator is a calcium channel blocker.

Pharmaceutical Composition 8: Pharmaceutical Composition 7, wherein the at least one calcium channel blocker is a dihydropyridine selected from the group consisting of nifedipine, isradipine, felodipine, amlodipine, nicardipine, and clevidipine.

Pharmaceutical Composition 9: Pharmaceutical Composition 7, wherein the at least one calcium channel blocker is a non dihydropyridine selected from the group consisting of verapamil and diltiazem.

Pharmaceutical Composition 10: Any One of Pharmaceutical Compositions 1 through 6, wherein the vasodilator is an ACE inhibitor.

Pharmaceutical Composition 11: Pharmaceutical Composition 10, wherein the ACE inhibitor is selected from the group consisting of benazepril, captopril, enalapril, fosinopril, lisinopril, moexipril, perindopril, quinapril, ramipril, and trandolapril.

Pharmaceutical Composition 12: Any One of Pharmaceutical Compositions 1 through 6, wherein the vasodilator is an angiotensin receptor blocker.

Pharmaceutical Composition 13: Pharmaceutical Composition 12, wherein the angiotensin receptor blocker is selected from the group consisting of azilsartan, candesartan, eprosartan, irbesartan, losartan, olmesartan, telmisartan, and valsartan.

Pharmaceutical Composition 14: Any One of Pharmaceutical Compositions 1 through 6, wherein the vasodilator is a nitrate.

Pharmaceutical Composition 15: Pharmaceutical Composition 14, wherein the nitrate is selected from the group consisting of nitroglycerin, isosorbide mononitrate and isosorbide dinitrate.

Pharmaceutical Composition 16: Any One of Pharmaceutical Compositions 1 through 6, wherein the vasodilator is an alpha blocker.

Pharmaceutical Composition 17: Pharmaceutical Composition 16, wherein the alpha blocker is selected from the group consisting of doxazosin, prazosin, and terazosin.

Pharmaceutical Composition 18: Any One of Pharmaceutical Compositions 1 through 6, wherein the vasodilator is a beta blocker.

Pharmaceutical Composition 19: Pharmaceutical Composition 18, wherein the beta blocker is selected from the group consisting of acebutolol, atenolol, bisoprolol fumarate, carvedilol, esmilol, labetalol, metoprolol tartrate, metoprolol succinate, nadolol, nebivolol, penbutolol sulfate, propranolol, sotalol, hydrochlorothiazide, and bisoprolol.

Pharmaceutical Composition 20: Any One of Pharmaceutical Compositions 1 through 6, wherein the vasodilator is hydralazine.

Pharmaceutical Composition 21: Any One of Pharmaceutical Compositions 1 through 6, wherein the vasodilator is an angiotensin receptor-neprilysin inhibitor.

Pharmaceutical Composition 22: Pharmaceutical Composition 21, wherein the angiotensin receptor-neprilysin inhibitor is sacubitril/valsartan.

Pharmaceutical Composition 23: Any One of Pharmaceutical Compositions 1 through 22, wherein the concentration of the vasodilator is about 0.0001 mg per ml to 1000 mg per ml, optionally 1 mg per ml to 10 mg per ml, optionally 1 mg per ml to 1000 mg per ml, optionally 5 mg per ml to 10 mg per ml, optionally 10 mg per ml, optionally 20mg per ml, optionally 30mg per ml, optionally 60mg per ml, optionally 90mg per ml, optionally 120mg per ml, optionally 180mg per ml, optionally 240mg per ml.

Pharmaceutical Composition 24: Any One of Pharmaceutical Compositions 1 through 2, wherein the concentration of the vasodilator is from about 0.0001% by weight to about 20% by weight, optionally about 0.01% by weight, optionally about 0.1% by weight, optionally about 1% by weight, optionally about 10% by weight, optionally about 20% by weight.

Method 25: A method for the treatment or prophylaxis of alopecia in a patient in need thereof, comprising: administering an effective amount of the pharmaceutical composition according to any one of Pharmaceutical Compositions 1 through 24 to a patient in need thereof.

Method 26: Method 25, wherein the patient is male.

Method 27: Method 25, wherein the patient is female.

Method 28: Method 25, wherein the composition is administered once a day, optionally two or more times a day, optionally once a week, optionally two or more times a week, optionally once a month, optionally two or more times a month, optionally a plurality of times a year.

Any of the features the above referenced pharmaceutical compositions, uses, and methods is applicable to any other pharmaceutical composition, use, or method identified herein. Moreover, any of the features of the above referenced pharmaceutical compositions, uses, and methods is independently combinable, partly or wholly, with other embodiments of the pharmaceutical compositions, uses, and methods described herein in any way, e.g., one, two, or three or more features may be combinable in whole or in part. Further, any of the features of the pharmaceutical compositions, uses, and methods described above may be made optional to other pharmaceutical compositions, uses, and methods described herein. Any aspect or embodiment of a method or use described herein can be performed using a composition, e.g., a pharmaceutical composition and/or a compound as described herein, and any aspect or embodiment of a composition, e.g., a pharmaceutical composition and/or a compound described herein, can be used or adapted to perform a method or use as described herein.

The above description presents the best mode contemplated for carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above that are fully equivalent. Consequently, this invention is not limited to the particular embodiments disclosed. On the contrary, this invention covers all modifications and alternate constructions coming within the spirit and scope of the invention as generally expressed by the following claims, which particularly point out and distinctly claim the subject matter of the invention. While the disclosure has been illustrated and described in detail in the drawings and foregoing description, such illustration and description are to be considered illustrative or exemplary and not restrictive.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ including but not limited to,' or the like; the term ‘comprising’ as used herein is synonymous with ‘including,’ containing,' or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article ‘a’ or ‘an’ does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases ‘at least one’ and “one or more” to introduce claim recitations. However, the use of such phrases should not be construed to imply that the introduction of a claim recitation by the indefinite articles ‘a’ or ‘an’ limits any particular claim containing such introduced claim recitation to embodiments containing only one such recitation, even when the same claim includes the introductory phrases ‘one or more’ or ‘at least one’ and indefinite articles such as ‘a’ or ‘an’ (e.g., ‘a’ and/or ‘an’ should typically be interpreted to mean ‘at least one’ or ‘one or more’); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of ‘two recitations,’ without other modifiers, typically means at least two recitations, or two or more recitations). Furthermore, in those instances where a convention analogous to ‘at least one of A, B, and C, etc.’ is used, in general such a construction is intended in the sense one having skill in the art would understand the convention (e.g., ‘a system having at least one of A, B, and C’ would include but not be limited to systems that have A alone, B alone, C alone, A and B together, A and C together, B and C together, and/or A, B, and C together, etc.). In those instances where a convention analogous to ‘at least one of A, B, or C, etc.’ is used, in general such a construction is intended in the sense one having skill in the art would understand the convention (e.g., ‘a system having at least one of A, B, or C’ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase ‘A or B’ will be understood to include the possibilities of ‘A’ or ‘B’ or ‘A and B.’

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

What is claimed is:

1. A pharmaceutical composition for the treatment or prophylaxis of alopecia, comprising:
at least one vasodilator; and
at least one pharmaceutical excipient.

2. The pharmaceutical composition of claim 1, in a form adapted for direct administration or subcutaneous injection to the scalp or epidermis.

3. The pharmaceutical composition of claim 2, wherein the form is selected from the group consisting of an oil, a liquid and a suspension for direct application on the scalp or epidermis.

4. The pharmaceutical composition of claim 1, formulated as a liquid or a suspension of the at least one vasodilator, wherein the vasodilator is a contact vasodilator.

5. The pharmaceutical composition of claim 1, wherein the vasodilator is a calcium channel blocker.

6. The pharmaceutical composition of claim 5, wherein the at least one calcium channel blocker is a dihydropyridine selected from the group consisting of nifedipine, isradipine, felodipine, amlodipine, nicardipine, and clevidipine.

7. The pharmaceutical composition of claim 5, wherein the at least one calcium channel blocker is a non dihydropyridine selected from the group consisting of verapamil and diltiazem.

8. The pharmaceutical composition of claim 1, wherein the vasodilator is an ACE inhibitor is selected from the group consisting of benazepril, captopril, enalapril, fosinopril, lisinopril, moexipril, perindopril, quinapril, ramipril, and trandolapril.

9. The pharmaceutical composition of claim 1, wherein the vasodilator is an angiotensin receptor blocker is selected from the group consisting of azilsartan, candesartan, eprosartan, irbesartan, losartan, olmesartan, telmisartan, and valsartan.

10. The pharmaceutical composition of claim 1, wherein the vasodilator is a nitrate is selected from the group consisting of nitroglycerin, isosorbide mononitrate and isosorbide dinitrate.

11. The pharmaceutical composition of claim 1, wherein the vasodilator is an alpha blocker is selected from the group consisting of doxazosin, prazosin, and terazosin.

12. The pharmaceutical composition of claim 1, wherein the vasodilator is a beta blocker is selected from the group consisting of acebutolol, atenolol, bisoprolol fumarate, carvedilol, esmilol, labetalol, metoprolol tartrate, metoprolol succinate, nadolol, nebivolol, penbutolol sulfate, propranolol, sotalol, hydrochlorothiazide, and bisoprolol.

13. The pharmaceutical composition of claim 1, wherein the vasodilator is hydralazine.

14. The pharmaceutical composition of claim 1, wherein the vasodilator is an angiotensin receptor-neprilysin inhibitor.

15. The pharmaceutical composition of claim 14, wherein the angiotensin receptor-neprilysin inhibitor is sacubitril/valsartan.

16. The pharmaceutical composition of claim 1, wherein the concentration of the vasodilator is about 0.0001 mg per ml to 1000 mg per ml.

17. A method for the treatment or prophylaxis of alopecia in a patient in need thereof, comprising:
administering an effective amount of the pharmaceutical composition according to claim 1 to a patient in need thereof.

18. The method of claim 17, wherein the patient is male.

19. The method of claim 17, wherein the patient is female.

20. The method of claim 17, wherein the composition is administered once a day, optionally two or more times a day, optionally once a week, optionally two or more times a week, optionally once a month, optionally two or more times a month, optionally a plurality of times a year.
